(19) United States
(12) Patent Application Publication
(10) Pub. No.: US 2026/0325843 A1
(43) Pub. Date: Oct. 22, 2026

(54) Electroacoustic Stimulation Device and Method

(71) Applicant: Zeev V. Kitov, Rockville, MD (US)

(72) Inventor: Zeev V. Kitov, ROCKVILLE, MD (US)

(21) Appl. No.: 19/644,454

(22) Filed: Apr. 10, 2026

Related U.S. Application Data
(63) Continuation-in-part of application No. 18/090,165, filed on Dec. 28, 2022, now Pat. No. 12,661,301.

(51) Int. Cl. A61N 1/36; A61H 23/02

(52) U.S. Cl. CPC A61N 1/36034; A61H 23/0245; A61N 1/36003; A61N 1/36028; A61H 2201/10

(57) ABSTRACT

A handheld electrostimulation device is disclosed that implements capacitive coupling electric field technology and dynamic electrode operation to generate time-varying and spatially non-uniform electric fields in biological tissue without galvanic electrical contact. The device includes a piezoelectric membrane comprising a piezoelectric ceramic layer bonded to a conductive plate and disposed within a dielectric enclosure. The ceramic layer is oriented toward target tissue and is driven by a low-voltage electrical signal at frequencies below mechanical resonance. Electrical excitation produces polarization of the piezoelectric material and establishes opposing electrical polarities on the ceramic layer and conductive plate, resulting in spatially distributed electric field projections and displacement currents within adjacent tissue. The device may be applied directly to the skin or through a dielectric layer.

### CROSS REFERENCE TO RELATED APPLICATIONS

This application is continuation in part claiming the benefit of U.S. Provisional Patent Application No. 63/294,479, filed Dec. 29, 2021, and Non-Provisional patent application Ser. No. 18/090,165 filed on Dec. 28, 2022, which are incorporated by reference herein in their entirety.

### FIELD OF THE INVENTION

The present invention relates to electroacoustic stimulation devices and methods, and more particularly to capacitive coupling electric field electrode technology employing a piezoelectric element that produces electric fields and associated mechanical vibrations in an acoustic frequency range. The invention is directed at noninvasive devices and methods that may be applied to the skin to provide sensory and neuromodulation using capacitive coupling and displacement currents.

In certain embodiments the device generates coordinated electric field and mechanical vibrations. The electric field interacts with tissue and neural structures beneath the skin promoting muscle relaxation, reduction of physical tension, and enhancement of comfort following physical activity. The invention further relates to methods of applying the device generating electric fields to the body in different orientations and configurations to provide localized or distributed effects and to support sports and fitness related muscle relaxation, pre-physical training activity, recovery after exertion, general relaxation of the body, stress reduction, and improvement of overall well-being and mood.

### BACKGROUND OF THE INVENTION

Vibrational, acoustic, and electrical stimulation modalities are widely used in sports, fitness, and therapeutic applications to promote muscle relaxation, reduce physical tension, and support recovery following physical activity. However, existing approaches exhibit limitations in energy efficiency, controllability, user comfort, and suitability for localized, repeatable stimulation.

Therapeutic ultrasound systems deliver acoustic energy into tissue, where absorption produces localized heating and mechanical effects. While such systems may provide therapeutic benefit, they typically require relatively high-power densities and specialized coupling media and may present a risk of tissue overheating if improperly applied. In addition, ultrasound systems are generally bulky and are not well suited for frequent, localized, or unsupervised use in consumer or wellness settings.

Mechanical vibration devices represent another commonly used modality. These devices generate oscillatory motion at the body surface to induce mechanical displacement of tissue and increase circulation. However, such systems rely on bulk mechanical energy transfer, typically require relatively large actuators, and provide limited ability to control the spatial distribution or depth of stimulation. Their effects are generally diffuse and lack precision.

Transcutaneous electrical nerve stimulation (TENS) devices apply electrical current through electrodes in direct conductive contact with the skin. These systems operate by injecting current into the body at amplitudes sufficient to depolarize peripheral nerve fibers. As a result, TENS produces forced neural activation rather than affects the endogenous activity.

Because TENS relies on conductive current injection, electrical current preferentially follows low-impedance pathways into deeper tissue regions, resulting in limited control over spatial distribution of stimulation. This often produces nonuniform current density, discomfort at the electrode interface, and unintended activation of motor fibers.

Furthermore, repeated conductive stimulation at fixed locations and parameters can lead to physiological adaptation. Such adaptation may involve reduced responsiveness of peripheral nerve fibers, diminished effectiveness of spinal gating mechanisms, and decreased perceptual response over time. As a result, stimulation intensity or parameters must often be increased to maintain effectiveness.

Accordingly, there remains a need for a compact, low-power, non-invasive device capable of delivering controlled and localized perturbation of tissue without requiring direct conductive contact, high acoustic power levels, or large mechanical actuators. There is further a need for a system that enables controlled interaction with superficial tissue structures while reducing discomfort, minimizing habituation, and improving repeatability of stimulation effects.

Early investigations have suggested that externally applied electric fields, when capacitively coupled to biological tissue without galvanic contact, may influence physiological and neurological responses. However, prior approaches have not provided effective mechanisms for controlling electric field distribution, enhancing coupling efficiency, or achieving spatially selective interaction with superficial tissue structures.

### SUMMARY OF THE INVENTION

The present disclosure relates to capacitive coupling electric field systems configured to project time-varying electric fields into biological tissue without galvanic electrical contact. In various embodiments, a piezoelectric membrane driven by an electrical excitation signal generates displacement-current-coupled electric fields that interact with superficial tissue layers through capacitive coupling.

The device includes a piezoelectric membrane comprising a piezoelectric ceramic layer bonded to a conductive plate, arranged such that the ceramic layer faces a target tissue during use. When electrically excited, the membrane produces a spatially bipolar electric field projection at a tissue interface, resulting in displacement currents within superficial tissue layers without conductive current injection.

Due to the dielectric properties of the stratum corneum and associated boundary conditions at the skin interface, the projected electric fields preferentially concentrate within superficial tissue regions, including the epidermis and immediately adjacent sub-epidermal structures. This interaction is governed primarily by displacement currents and electric field gradients rather than bulk conductive current flow.

In certain embodiments, the device further comprises at least one passive spatial field shaping (focusing) element positioned adjacent to the piezoelectric membrane. Such elements, including conductive rings, perforated plates, or shaped conductive structures, are configured to selectively redistribute electric field lines to concentrate field intensity at predetermined regions of the tissue interface.

In certain embodiments, a controlled displacement-current return path is established through capacitive coupling to a user and surrounding environment, thereby stabilizing electric field distribution without conductive grounding.

In additional embodiments, the device implements Dynamic Electrode Technology (DET), in which mechanical vibration of the piezoelectric membrane produces time-varying changes in electrode geometry and position. Such motion results in lateral displacement of electric field maxima across the tissue surface, enabling sequential stimulation of spatially distinct microdomains.

In further embodiments, the device operates in a floating electrical configuration in which electrodes are galvanically isolated from circuit ground. A displacement-current return path is established through distributed capacitive coupling between the device, the user, and the surrounding environment, thereby stabilizing electric field distribution without direct conductive current flow.

In conventional piezoelectric actuator systems, external fringe electric fields are typically regarded as parasitic and are minimized or shielded. In contrast, the present invention intentionally utilizes and enhances such external electric fields as a primary functional mechanism for capacitive coupling into biological tissue.

The disclosed system enables controlled influence of tissue electrical conditions through spatially and temporally varying electric fields, with interaction primarily governed by electric field gradients rather than absolute voltage magnitude.

This approach provides localized, non-contact stimulation of superficial tissue structures with reduced discomfort and improved stability compared to conductive stimulation systems.

### BRIEF DESCRIPTION OF THE DRAWINGS

FIG. 1A is a top plan view of the capacitive coupling electric field stimulation device in accordance with the present invention.

FIG. 1B is a bottom plan view of the capacitive coupling electric field stimulation device in accordance with the present invention.

FIG. 2 is a general elevational cross-sectional view of the capacitive coupling electric field stimulation device in accordance with the present invention.

FIGS. 3A and 3B are enlarged elevational cross-sectional views of the vibrating housing and the attached bottom active wall in accordance with a preferred embodiment of the present invention.

FIG. 4A is a block diagram of an electrical circuit that employs an enhanced supply voltage.

FIG. 4B is a block diagram of an electrical circuit that uses an output transformer as an enhancement element.

FIG. 5 is an electrical schematic diagram of a signal adjustment circuit that provides signal biasing.

FIG. 6 is an electrical schematic diagram of another version of a signal adjustment circuit that produces a biased signal.

FIG. 7 is a block diagram of an adjustment circuit in the form of a voltage multiplier.

FIG. 8 presents the spectral composition of a frequency modulated signal.

FIG. 9 is a block diagram of an electrical circuit generating signal which is frequency and amplitude modulated.

FIG. 10 illustrates different modes for positioning the piezoelectric element relative to the skin surface.

FIG. 11 shows the piezoelectric membrane forming Dynamic Field Redistribution of the electric field.

FIG. 12 shows implementations of driving circuits with floating electrode arrangement.

FIG. 13 demonstrates General Reaction Coefficient vs. time in experimental setting.

FIG. 14 Equivalent Circuit Modeling of the Skin-Electrode Interface.

FIG. 15 Equivalent circuit representation of the viable epidermis.

### DETAILED DESCRIPTION OF THE PREFERRED EMBODIMENT

### Definitions

To clarify elements of the disclosure, several definitions are provided below.

Cutaneous Capacitive Coupling Modulation (3CM) Technology refers to a process in which time-varying electric fields generated by an external electrode assembly capacitively couple across an intervening dielectric interface with a biological surface, thereby inducing displacement currents within cutaneous tissue without intentional galvanic current delivery. Such coupling enables changes in biological activity through electric field interaction rather than through conductive current injections.

Dynamic Electrode Technology (DET) refers to a field-generating architecture in which an electrically excited electrode assembly undergoes mechanical vibration, resulting in time-varying changes in electrode geometry and position relative to a biological interface. Such motion produces a time-varying spatial redistribution of the projected electric field at the tissue surface, including lateral displacement of regions of maximum field intensity and variation in the angular orientation of electric field lines.

Electric fields of alternating polarity and varying magnitude are thereby projected onto the skin in a dynamically shifting pattern, enabling sequential interaction with spatially distinct regions of the tissue interface. Modulation, as used herein, refers to an influence on endogenous electrical or physiological activity within biological tissue produced by externally applied electric fields, without direct conductive current injection or deterministic induction of action potentials.

In this context, modulation encompasses alteration of electrical conditions, membrane polarization states, or neural activity patterns arising from capacitive coupling and displacement-current interaction. The term “modulation” is used to describe biological or physiological effects and is distinct from signal modulation (e.g., frequency or amplitude modulation).

As used herein, the terms “floating electrode,” “floating electrode system,” or “electrically floating” refer to an electrode or electrode assembly that is galvanically isolated from the device power supply and ground reference, such that no direct conductive (DC) current path exists between the electrode and circuit ground.

A floating electrode may nonetheless be capacitively coupled to one or more circuit nodes through discrete capacitors, parasitic capacitances, transformer windings, or other displacement-current pathways. Such capacitive coupling permits transmission of time-varying electric fields and displacement currents while maintaining galvanic isolation. Accordingly, an electrode described as “floating” within this specification should be understood to be:
Galvanically non-referenced (no conductive path to ground), and
Capacitively or by displacement-current coupled to driving circuitry.

The presence of capacitive elements in the drive circuitry therefore does not negate the floating nature of the electrode system but rather defines the mechanism by which excitation fields are coupled while preserving galvanic isolation.

A floating configuration of the driving signals refers to a mode in which the piezoelectric element is driven by two identical but counter-phase signals. The voltage potential difference between two signals driving the piezoelectric element is neither coupled to, nor referenced to, the ground potential of the electronic circuit.

Floating reference point is a node in the driving circuit, which is neither coupled to, nor referenced to the ground potential or other fixed potential of the electronic circuit.

A piezoelectric element is any piezoelectric device that converts an electrical signal into vibrations and a corresponding electric field. In the present invention, the piezo element is preferably a piezo bender type device.

A piezoelectric membrane is a combination of the piezo-ceramic disk and the metal disk sandwiched together.

A significant sideband in a frequency-modulated signal refers to a spectral component whose amplitude or power is sufficient to contribute materially to the composite waveform and to the resulting electric field generated by the electrode assembly.

In certain embodiments, sidebands may be considered significant when their amplitude exceeds a predetermined fraction of the carrier amplitude, for example on the order of approximately 0.1 or greater, such that the sideband carries a non-negligible portion of signal energy and contributes to electric field formation at the biological interface.

Coherent signals of different frequencies are signals that maintain a constant, well-defined phase relationship with one another.

The sideband index N indicates the relative frequency displacement of each individual sideband element from the carrier frequency.

Deep sub-resonance” refers to an operating regime of a piezoelectric membrane in which an applied electrical drive signal has a frequency substantially below a mechanical resonance frequency of the membrane, such that mechanical vibration is minimal or negligible and electric field coupling is predominantly capacitive.

Capacitive coupling between all elements of the device including electrodes, focusing elements, return path conductive plate and some nodes of the drive circuit may be established by a capacitor component electrically connected between them. The capacitor component may be a discrete or integrated element configured to provide a defined capacitance. This definition is intended to exclude equivalent or distributed capacitances inherent in other electrical components, including but not limited to the parasitic capacitance of transistors, resistors, electrodes, conductive plates, and other electronic parts.

### Apparatus-Tissue Interactions

Our experiments [1], [2] demonstrated that the capacitive-coupling electric-field modulation device of the present invention can produce measurable physiological and neurological effects even when isolated from the subject body or positioned at a distance from the subject's body without direct conductive contact.

Some other biologic effects produced by externally applied electric fields was also demonstrated in prior art. U.S. Pat. No. 5,782,874 teaches that electric fields capacitively coupled to the body may influence the peripheral nervous system.

The scientific basis for presented inventive methods is well documented. Due to the dielectric dominance of superficial epidermal layers and the capacitive boundary conditions present at the skin interface, the projected electric fields preferentially concentrate within the stratum corneum and viable epidermis. Tissue dielectric spectra and frequency-dependent impedance transitions governing such displacement-current coupling have been characterized in the literature [Gabriel et al., pp. 2273-2278].

Modeling studies further demonstrate that electric field gradients and penetration profiles are strongly dependent on electrode geometry and return-path configuration [Miranda et al., pp. 1623-1630]. Additionally, superficial impedance variability associated with sweat duct pathways and dynamic skin conductance behavior has been described. Birgersson Thesis, pp. 33-41].

It is also known that a piezoelectric element, when electrically activated, produces not only an internal electric field which causes bending of the attached conductive plate, but also an external or fringe electric field extending into the surrounding space. In conventional piezoelectric actuator design, such external fringe fields were generally regarded as parasitic or detrimental because they represent energy dissipation outside the intended electromechanical conversion path. Considerable engineering effort was therefore directed toward shielding, grounding, or otherwise minimizing these external fields.

In contrast, the present invention intentionally utilizes and enhances the external or fringe electric field component of the piezoelectric structure. Rather than suppressing the fringe field, the invention exploits this field as a primary functional modality for non-contact or near-contact modulation of tissue electrical activity.

Prior-art teachings, including those of U.S. Pat. No. 5,782,874, further establish that when conductive plates separated by a dielectric medium and maintained at different electric potentials are positioned in proximity to the subject's body, the resulting external electric field induces capacitive coupling into the subject's tissues and may evoke neurological responses.

However, it is well known that at relatively low operating frequencies, capacitive coupling alone induces displacement currents and associated potentials that are typically below the threshold required for classical nerve depolarization or direct action-potential triggering. Despite this subthreshold magnitude, such fields have been observed to produce measurable responses within the central nervous system.

Since classical nerve stimulation does not occur under these conditions, the induced signals are believed to operate through stochastic modulation of spontaneous neural firing patterns rather than deterministic excitation.

However, employing subthreshold stochastic modulation presents substantial technical challenges. The induced electric potentials and displacement currents are extremely small and must compete with endogenous bioelectrical activity and thermal noise whose amplitudes may substantially exceed the induced signal levels. Consequently, the reliability and reproducibility of purely capacitive coupling effects depend on the field strength, spatial coherence, signal spatial gradient, spectral richness, and coupling efficiency.

To address these obstacles, the present invention introduces multiple electric field enhancement and field shaping strategies, which may be used individually or in combination, including but not limited to:
1. Formation of a bipolar or double-pole electric field directed toward the tissue to form local spatial field gradients thus enhancing displacement current density.
2. Spatial field scanning, such as dynamic tilting, oscillating, or angularly displacing the primary conductive member, for example vibrating metal disk 21, thereby producing time-varying shifting field projection patterns onto the tissue.
3. Spatial Field Shaping, including the use of focusing conductive structures such as grids, rings, perforated plates, or shaped electrodes configured to concentrate or redistribute electric field lines.
4. Spectral enrichment of the electric field frequency band by employing frequency modulation or other related methods, thereby generating multiple sideband components and increasing the probability of interaction with frequency-sensitive biological processes.
5. Establishment of a controlled return-path coupling mechanism to complete the displacement currents flow via capacitive elements through the subject's body and surrounding environment, thereby stabilizing displacement current flow and enhancing the process reliability.
6. Operation in a floating electrical configuration, wherein the field-generating structure is not galvanically tied to earth ground or any reference potential, allowing dynamic potential redistribution and enhanced capacitive interaction with the subject.

Collectively, these measures increase the effective coupling of externally generated electric fields into biological tissues and improve the efficacy of subthreshold neuro-modulatory interaction.

### 1. Stratified Electrical Structure of the Tissue

The skin and its superficial neural structures present a stratified electrical medium comprising layers having distinct dielectric constants, conductivities, and capacitive properties.

In one non-limiting anatomical model, the tissue region interacting with the externally projected electric field includes Stratum corneum, Viable epidermis, Dermis, and Superficial vascular and neural plexuses.

The top layer of the skin, the stratum corneum (SC) exhibits relatively low conductivity and high dielectric impedance due to its keratinized cellular structure and reduced water content. This layer therefore behaves predominantly as a dielectric barrier through which displacement currents preferentially pass under capacitive coupling conditions. At the same time the stratum corneum substantially blocks passage of galvanic currents.

Beneath the stratum corneum, the viable epidermis contains hydrated cellular matrices, intercellular fluids, and membrane-bound structures that provide both resistive and capacitive pathways.

This layered impedance structure results in non-uniform electric field distribution when exposed to externally generated electric fields.

### 2. Membrane Polarization and Superficial Displacement-Current Coupling

Cellular membranes constitute lipid bilayer dielectric structures separating conductive intracellular and extracellular media. When exposed to time-varying electric fields, these membranes undergo polarization due to charge displacement across the membrane thickness. Such polarization does not require net charge transfer across the membrane and may occur under purely capacitive coupling conditions.

Under non-contact electric field exposure, coupling into biological tissue is predominantly capacitive, and the induced currents within the tissue are primarily displacement currents rather than conductive or galvanic currents. Due to the high impedance of the stratum corneum and the presence of an intervening dielectric interface, electric field energy is preferentially concentrated within superficial tissue layers.

This results in enhanced electric field gradients in upper epidermal regions and reduced penetration of conductive current into deeper tissues. Consequently, interaction with tissue is localized primarily to superficial cellular and neural structures.

At microscopic scales, membrane capacitance is several orders of magnitude greater than bulk tissue capacitance. Accordingly, localized electric field gradients and associated displacement currents may induce polarization of membrane structures even when macroscopic current levels remain below classical stimulation thresholds.

Relevant electrically responsive structures include keratinocyte membranes, fibroblast membranes, vascular endothelial membranes, free nerve ending membranes, and Schwann cell sheaths.

This mode of interaction differs from conductive stimulation systems in which injected current preferentially follows low-resistance pathways into deeper tissue regions.

### 3. Neural and Microstructural Interaction Pathways

Superficial tissue layers contain multiple electrically sensitive neural elements, including free nerve endings, C-fiber nociceptors, A-delta fibers, mechanoreceptive terminals, and autonomic neurovascular interfaces. These neural structures exhibit spontaneous baseline firing patterns and membrane excitability states that may be influenced by externally applied electric fields, including under conditions in which field amplitude remains below classical depolarization thresholds. Such influence may manifest as stochastic variation in firing probability rather than deterministic action potential triggering.

Skin appendages introduce localized impedance discontinuities that affect electric field distribution within superficial tissue. These structures include sweat ducts, hair follicles, and sebaceous gland channels.

Sweat ducts, in particular, form helically shaped conductive microchannels that traverse the stratum corneum. Due to their electrolyte content and geometry, these ducts may act as localized field concentrators under externally applied electric fields, enhancing local electric field gradients and displacement-current coupling.

The density and spatial distribution of such appendageal structures may reach hundreds per square centimeter, resulting in a network of spatially distributed micro-coupling sites through which electric field interaction with neural and cellular elements is preferentially mediated.

### 4. Field Gradient Sensitivity

Biological response to externally applied electric fields is influenced not only by absolute field magnitude but also by spatial and temporal gradients of the field. In particular, localized variations in field intensity, orientation, and distribution contribute to interaction with electrically sensitive microstructures within superficial tissue layers.

The present invention produces time-varying electric field distributions characterized by changing field orientation, lateral displacement of regions of maximum field intensity, localized gradient intensification, and multi-frequency field components. These characteristics increase the probability of interaction with cellular membranes, neural elements, and appendageal structures.

Structural and operational features of the device contribute to these effects as follows:
1. Dynamic electrode motion alters electrode geometry and position, producing time-varying projection angles and lateral shifting of electric field gradients across superficial tissue layers.
2. Bipolar electric field configuration establishes opposing polarities at the tissue interface, increasing local potential gradients and associated displacement-current density.
3. Spatial field shaping, focusing elements, including rings, grids, and perforated conductive structures, redistribute electric field lines to concentrate field intensity at selected regions of the tissue interface.
4. Multi-frequency excitation, including frequency variation and associated sideband components, introduces multiple electric field components that interact with tissue through frequency-dependent coupling pathways.
5. Floating electrical operation enables adaptive charge redistribution relative to the impedance of the user's body and surrounding environment.
6. Distributed return-path coupling stabilizes displacement-current flow through superficial and appendageal pathways.

Without being bound to a specific biological mechanism, it is believed that the externally projected electric fields interact with superficial tissue structures through capacitive coupling and membrane polarization, resulting in stochastic modulation of endogenous neural activity. Such interaction occurs without requiring direct conductive current injection or deterministic nerve depolarization.

### Preferred Embodiment

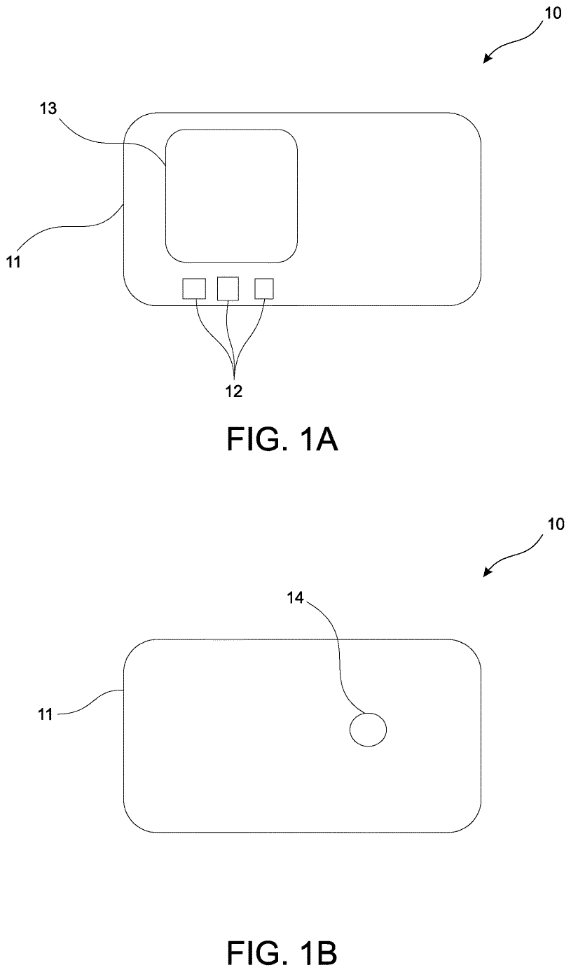

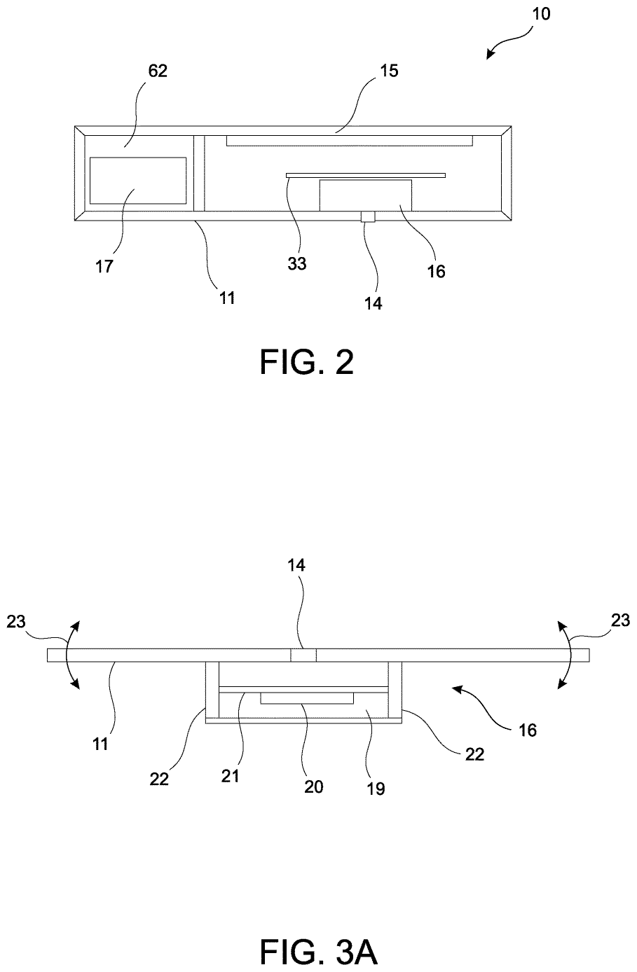

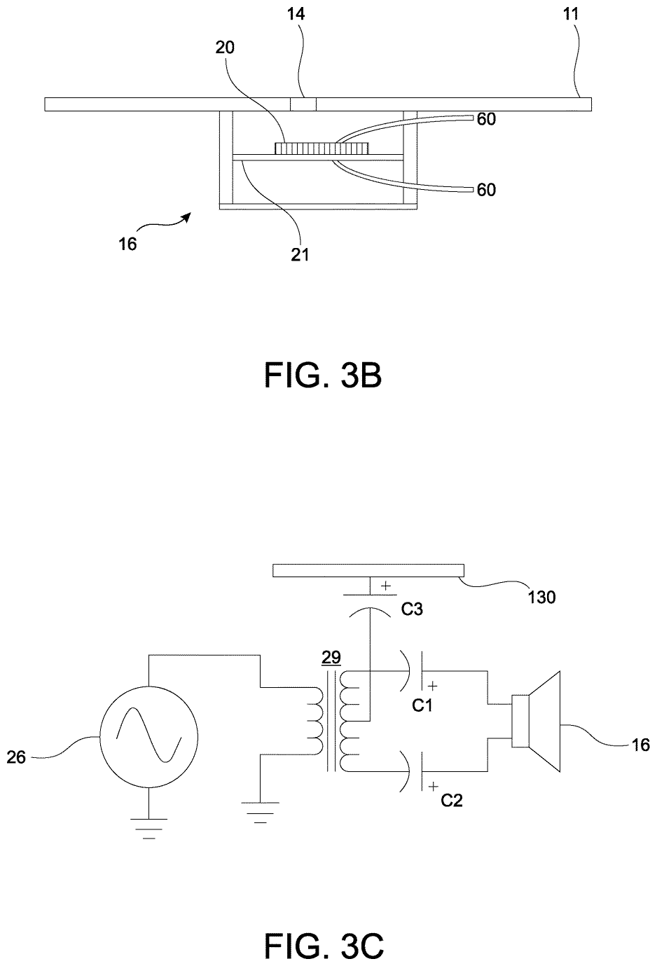

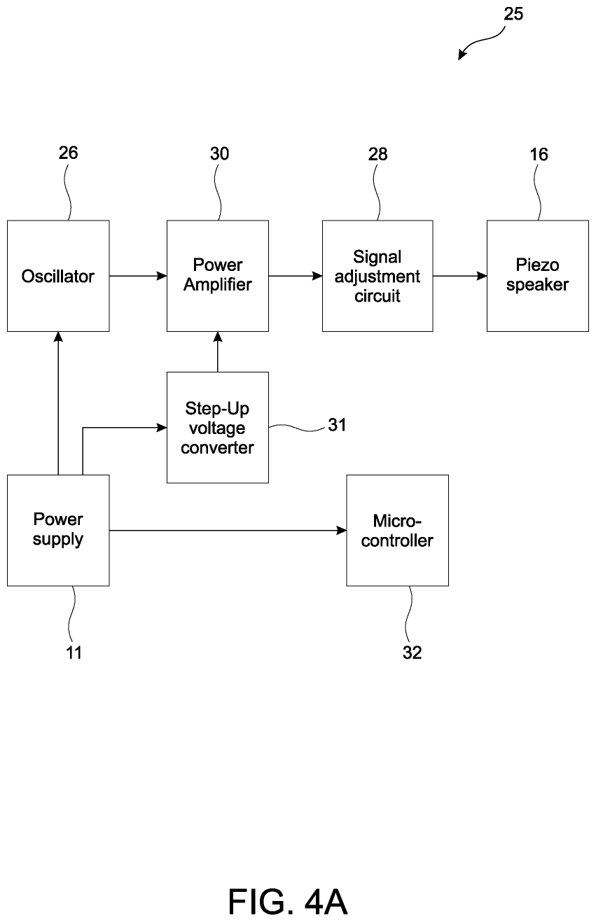

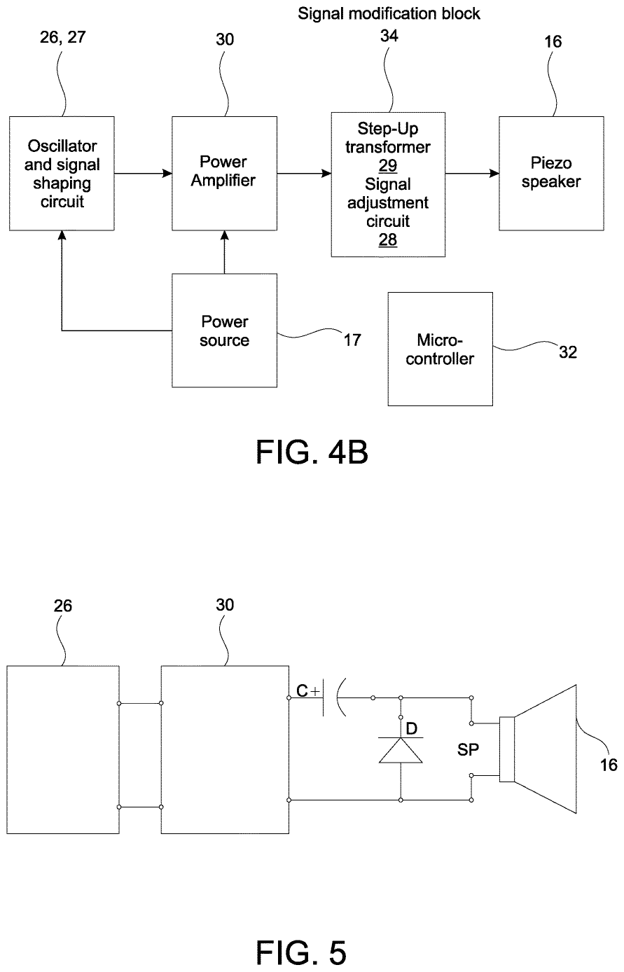

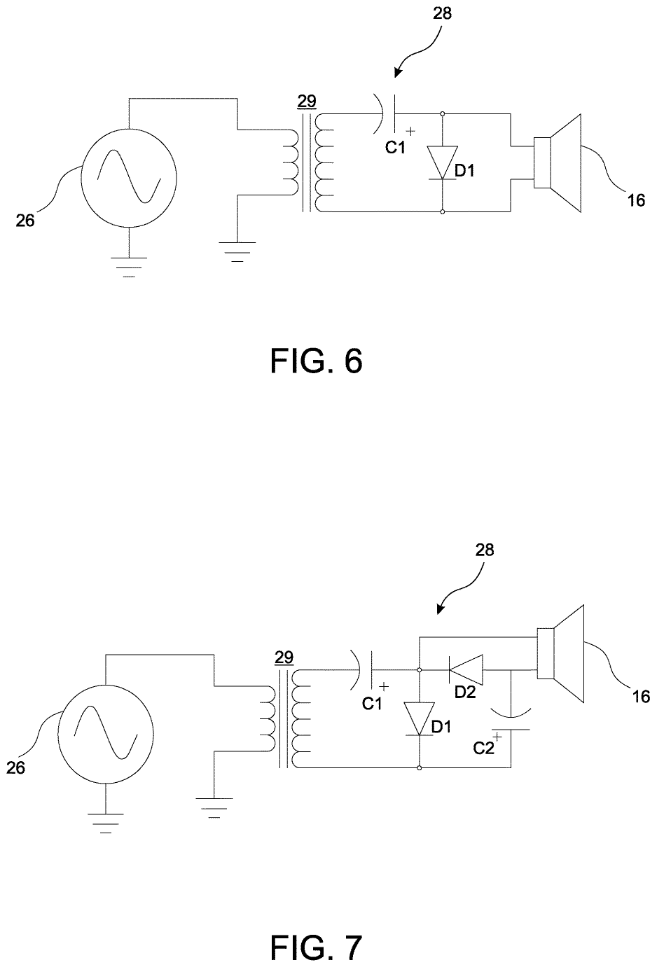

With reference to FIGS. 1-7, an example embodiment of the electric field stimulation device is illustrated.

The device comprises a housing containing a piezoelectric membrane including a piezoelectric ceramic element bonded to a conductive metal plate. The piezoelectric membrane is positioned such that, during use, the ceramic element faces the target tissue.

This orientation enhances projection of the electric field generated by the ceramic element toward the tissue interface. In contrast to conventional configurations in which the conductive plate faces outside and partially shields the electric field generated by the ceramic, the present configuration exposes the ceramic-facing field, resulting in increased electric field strength within superficial tissue layers and improved symmetry of the spatially bipolar electric field projection.

Electrical excitation of the piezoelectric membrane produces opposite electrical polarities on the ceramic element and the conductive plate, thereby generating a spatially bipolar electric field extending beyond the device and capacitively coupling to the tissue.

The device further includes an electrical drive circuit configured to apply a time-varying electrical signal across the piezoelectric membrane. In certain embodiments, the drive circuit comprises an oscillator and an amplification stage configured to provide a signal of alternating polarity. Signal amplitude may be increased when needed, using a step-up voltage converter or a transformer.

In some embodiments, the output stage of the drive circuit is electrically isolated from the power supply, for example through a transformer-coupled configuration, such that the piezoelectric membrane is driven in a floating electrical condition.

To enhance coupling efficiency, the device may further include a conductive member in the form of a conductive plate (33) disposed behind the piezoelectric element and oriented generally parallel to the piezoelectric element. The plate (33) may be electrically connected to, or capacitively coupled via one or more capacitors to, an electrode of the piezoelectric membrane, for example the conductive metal plate.

During operation, the conductive plate (33) functions as a distributed counter-electrode and modifies electric-field boundary conditions within and around the enclosure. Capacitive coupling between the conductive plate and external objects, including the user's hand during handheld operation, contributes to formation of a displacement-current return path.

This distributed return-path coupling stabilizes the electric field distribution and reduces sensitivity to uncontrolled environmental capacitance while maintaining minimal conductive current flow.

In one embodiment, the conductive plate (33) is positioned on an inner wall of the enclosure and is capacitively coupled through a capacitor having a capacitance in the range of approximately 0.1 μF to 10 μF. In a preferred configuration, the plate is coupled to the rear electrode (metal disk) of the piezoelectric membrane and functions to reduce common-mode drift of the electrode system.

When the ceramic side of the piezoelectric membrane, for example piezo-ceramic disk 20, is oriented toward the skin, the applied excitation signal produces a stronger outwardly directed electric field because the electric field generated by the ceramic element is no longer substantially shielded by the conductive metal disk 21. Such orientation is unconventional, as in most piezoelectric devices the metal disk faces the tissue to protect the mechanically fragile ceramic layer.

Because the ceramic disk 20 and the metal disk 21 are driven with opposite polarities, the resulting electric field is spatially bipolar, forming a polarity-divergent field distribution. This configuration also improves polarity symmetry of the electric field at the tissue interface, as both polarities are more evenly expressed.

The control electronic circuit (blocks 26, 34, 27, and 35) generates a sinusoidal or quasi-sinusoidal waveform having a frequency in the range of approximately 220 Hz to 400 Hz. Alternatively, the circuit may generate triangular or square waveforms within a similar frequency range.

### Floating Configuration

When the signal driving the piezoelectric element is derived from dual outputs of an amplifier or from a transformer secondary winding isolated from ground, the drive signal is electrically floating with respect to the device power supply.

Although the absence of galvanic contact between the electrodes and the skin inherently results in capacitive coupling at the electrode-tissue interface, electrical isolation of the electrode system from the device reference establishes a fully floating drive topology. In this configuration, the electrode pair is not constrained by a fixed potential relative to the power supply.

The floating configuration may be established through transformer isolation or by capacitive coupling between the electrodes and other conductive elements on one side and a reference node of the electronic circuit on the other side. Such capacitive coupling may be implemented through inclusion of one or more capacitor components, thereby permitting displacement-current coupling while maintaining galvanic isolation.

Conversion from a referenced (non-floating) configuration to a floating configuration permits symmetric potential excursions of the electrode pair relative to the surrounding environment. When bipolar signals are applied across spatially separated electrodes under floating conditions, the instantaneous differential potential between the electrodes may be increased relative to a single-ended referenced excitation derived from the same supply.

In a first-order approximation, the electric field magnitude between electrodes may be expressed as:

E≈V_diff/d

where V_diff represents the instantaneous differential electrode potential and d represents the effective separation distance through intervening dielectric and biological media. As V_diff increases under bipolar floating excitation, the resulting electric field gradient correspondingly increases.

Floating bipolar excitation also alters the geometry of the projected electric field. In a single-ended referenced configuration, field lines extend from the active electrode toward a distributed reference, producing a relatively diffuse field distribution. In contrast, bipolar floating excitation establishes the electric field gradient primarily between the electrode pair, resulting in a more spatially confined field structure characterized by steeper gradients and increased local field divergence.

In layered skin structures exhibiting dielectric dominance in the stratum corneum, such bipolar floating excitation promotes displacement-current coupling within superficial tissue layers without requiring galvanic charge transfer across the interface. This configuration reduces conductive current injection and favors interaction through displacement-current mechanisms.

Floating operation does not necessarily increase absolute penetration depth of the electric field; rather, it improves control of field symmetry, spatial distribution, and gradient formation. As a result, selectivity and stability of superficial tissue interaction may be enhanced relative to a referenced configuration, thus making this stochastic biologic effect more reliable.

The foregoing description illustrates functional differences between referenced and floating drive topologies and is not intended to limit the invention to any voltage, amplitude, waveform, isolation method, or return-path configuration.

### Return Path

Electric field lines originate on charges present on the electrodes and terminate on induced charges within the tissue and the surrounding environment. In such systems, continuity of the current loop is maintained through displacement currents rather than through direct conductive current flow. This principle enables capacitive coupling to biological tissue and permits effective operation of floating electrode configurations without direct current injections.

In a closed displacement-current loop, capacitive coupling occurs at multiple locations. A first capacitive interface is formed between the electrodes and the skin surface. A second capacitive interface is formed between the skin and the remainder of the return path, which may include distributed coupling to the environment, coupling to nearby conductive structures such as indoor power wiring, or coupling through the user's body back to the device.

Although this return portion of the loop is generally less dominant than the primary electrode-skin interface, it introduces an impedance that affects stability and repeatability of the induced displacement currents. Accordingly, controlled formation of the return path is desirable.

To stabilize the return path, a floating reference node may be provided. In one embodiment, a midpoint of a transformer secondary winding is coupled to a conductive plate (130), either directly or through a capacitor component. The conductive plate functions as a floating reference electrode.

The conductive plate may be positioned within the device housing, for example adjacent to an interior wall, to provide a sufficient surface area for capacitive coupling to the user's hand during operation. In this configuration, the user's body participates in the return path through distributed capacitive coupling.

In an alternative embodiment, the conductive plate may be positioned on an exterior surface of the housing. In this arrangement, the user may establish capacitive coupling or partial galvanic contact with the plate, depending on grip conditions. Even in the absence of strong conductive contact, the resulting capacitive coupling provides a more stable and controlled return path than configurations lacking a dedicated reference plate.

The conductive plate may be formed from electrically conductive materials including metals such as copper, aluminum, or steel, or from conductive polymers or composite materials.

When the conductive plate is coupled to its reference node through a capacitor component, direct current coupling is blocked and only alternating-current capacitive coupling is maintained. The coupling capacitor may be selected to have a capacitance substantially greater than the effective capacitance of the distributed return path, which is typically on the order of picofarads. For example, capacitance values in the microfarad range may be used.

Such a configuration stabilizes the displacement-current return path without materially impairing its operation and reduces sensitivity to environmental variability.

### Frequency Band Enrichment

The device employs frequency variation of the excitation signal to produce enrichment of the effective frequency band delivered to the piezoelectric element and coupled to biological tissue.

In frequency-modulated operation, the excitation signal comprises a carrier frequency and a plurality of spectral components distributed about the carrier. These components, commonly referred to as sidebands, are spaced from the carrier by integer multiples of the modulation frequency.

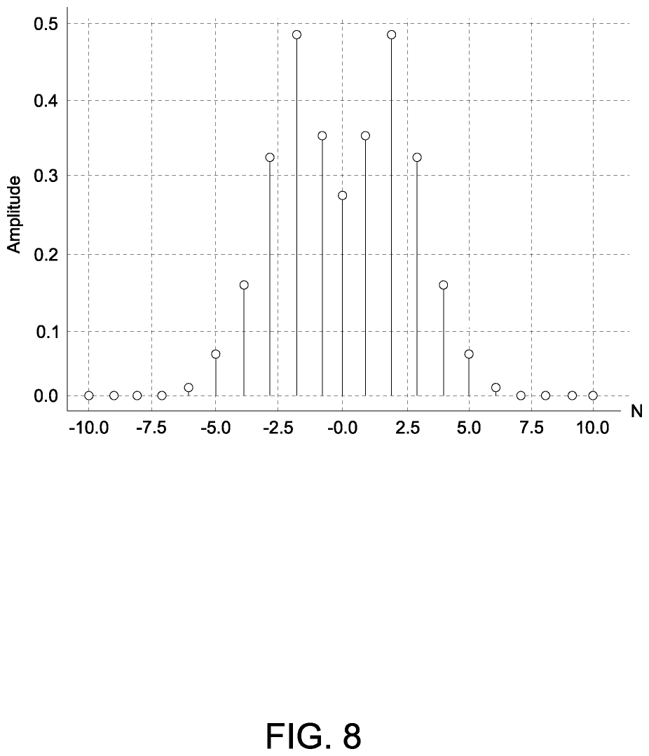

As illustrated in FIG. 8, frequency variation produces multiple sideband components distributed symmetrically around the carrier frequency. The number, amplitude, and distribution of these sidebands depend on the carrier frequency, peak frequency deviation, modulation frequency, and waveform of the modulating signal.

The extent of frequency variation may be characterized by a modulation index defined as:

m=Δf/fₘ

where Δf is the peak frequency deviation and fₘ is the modulation frequency. In certain embodiments, the modulation index is selected to produce a plurality of significant sidebands within the operating frequency band.

An additional parameter, referred to as the frequency change coefficient (FCC), may be defined as:

FCC=(Δf×100)/f_c

where f_c is the carrier frequency. The FCC represents the percentage variation of the carrier frequency and provides an alternative measure of frequency variation.

In certain embodiments, the device operates with carrier frequencies in the range of approximately 220 Hz to 400 Hz, with frequency variation applied by a lower-frequency signal in the range of approximately 0.1 Hz to 20 Hz. Such operation may produce a large number of significant spectral components confined within a relatively narrow band centered around the carrier.

As a result, the piezoelectric element is excited simultaneously at multiple closely spaced frequencies rather than at a single frequency. The electric field generated by the device therefore exhibits composite spectral content corresponding to both the carrier and associated sidebands.

Because biological tissue exhibits frequency-dependent electrical properties, different spectral components may couple differently into tissue structures, appendages, and membrane interfaces. The presence of multiple spectral components increases the likelihood of effective interaction with localized tissue inhomogeneities.

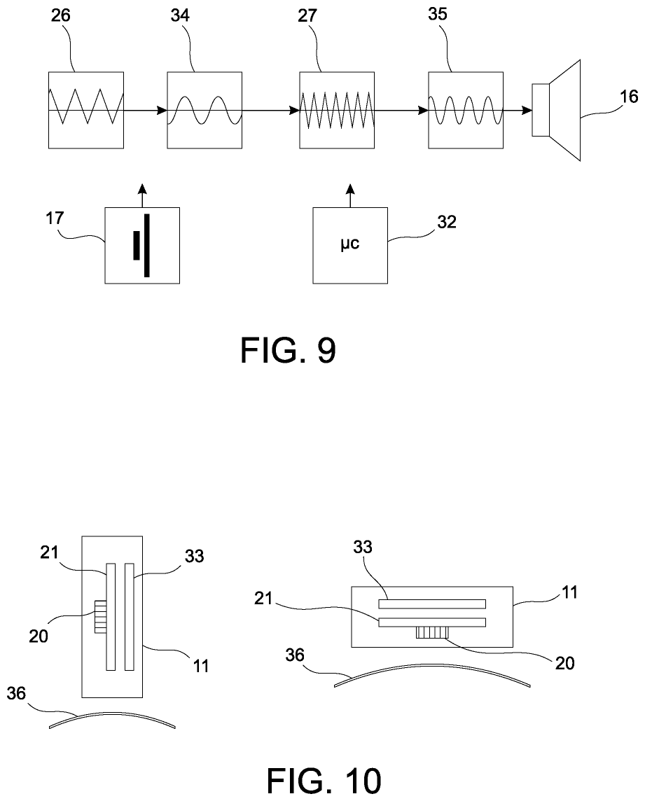

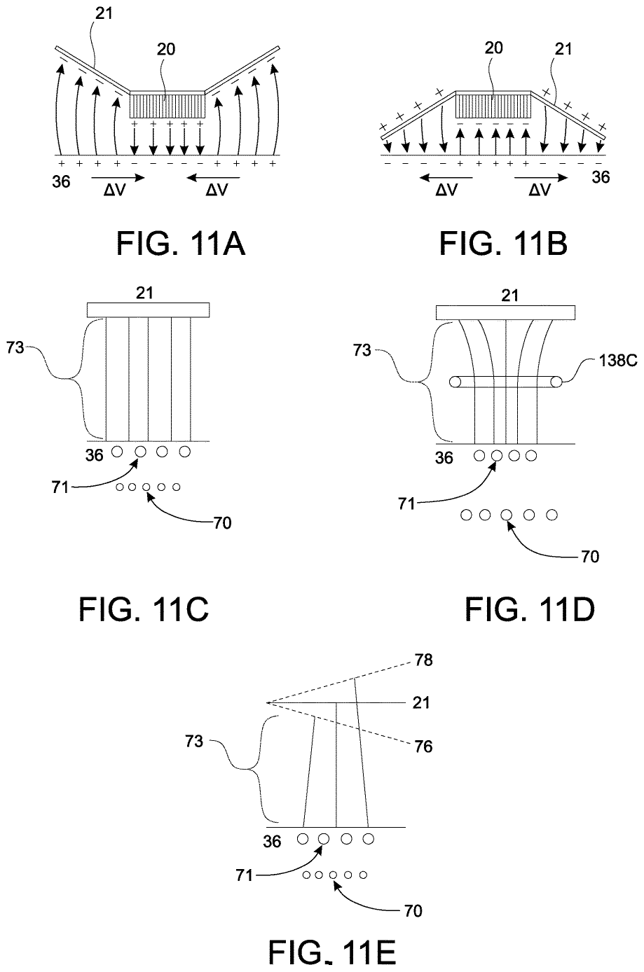

In certain implementations, frequency variation may be accompanied by amplitude variation arising from frequency-dependent signal conditioning. As illustrated in FIG. 9, such amplitude variation may result in an effective amplitude envelope superimposed on the excitation signal.

The described approach increases spectral diversity of the applied electric field without requiring an increase in overall signal amplitude.

### Biological Effects of Frequency Variation

In one exemplary embodiment, the device employs a frequency-varying excitation signal having a carrier frequency of approximately 250 Hz, a modulation frequency of approximately 1 Hz, and a high modulation index. This produces a set of phase-coherent spectral components at frequencies of approximately 250 Hz±n·1 Hz for multiple integer values of n, resulting in a slowly varying instantaneous frequency. The resulting spectral structure represents controlled spectral spreading rather than broadband noise.

Frequency variation does not materially alter the depth distribution of the electric field within biological tissue. Because the spectral components remain near the carrier frequency and tissue impedance varies only gradually over this range, the system operates in a quasi-static regime. Accordingly, the electric potential distribution across tissue depth remains substantially similar to that produced by a single-frequency excitation at the carrier frequency.

Despite this, frequency variation may enhance interaction with biological tissue. The presence of multiple closely spaced spectral components produces temporal variation in local electric field conditions at the tissue interface. Such variation may reduce steady-state accommodation effects and increase interaction with electrically responsive structures, particularly under low-amplitude field conditions.

Frequency variation may further enhance the effects of spatial electric field redistribution produced by Dynamic Electrode Technology (DET). Electrode geometry establishes spatial gradients of electric potential, and the presence of multiple spectral components causes these spatial patterns to evolve over time. This may result in time-varying distributions of electric field gradients and associated changes in local electrical conditions within tissue microdomains.

In addition, the presence of multiple spectral components enables interaction with biological structures exhibiting different electrical time constants. Nerve fibers, cellular membranes, ion channels, and intercellular junctions may respond differently to distinct spectral components. The resulting spectral diversity may increase the likelihood of effective interaction across a range of tissue structures without requiring tuning to a single frequency.

In certain embodiments, the modulation frequency is selected in the range of approximately 0.1 Hz to 20 Hz. Lower modulation frequencies, including frequencies on the order of approximately 1 Hz, produce slow temporal variation of the excitation signal that may correspond to time scales of endogenous physiological processes. Such operation may support cumulative effects over time without requiring high instantaneous field amplitudes.

Without being bound to a specific biological mechanism, it is believed that frequency variation contributes to stochastic modulation of endogenous electrical activity within superficial tissue structures through capacitive coupling and membrane polarization.

### Amplitude Variation (Envelope Modulation)

In certain embodiments, the excitation signal applied to the piezoelectric element includes amplitude variation in addition to frequency variation. The amplitude variation introduces a lower-frequency envelope superimposed on a higher-frequency carrier signal.

Amplitude variation may be characterized by a modulation index defined as:

m=(A_max−Amin)/(A_max+A_min)

where A_max and A_min represent the maximum and minimum amplitudes of the modulated signal, respectively. In certain embodiments, the modulation index is within a range of approximately 0.1 to 0.7.

Amplitude variation produces a carrier signal accompanied by a pair of spectral components positioned symmetrically around the carrier frequency. These components are coherent with the carrier and define a slowly varying amplitude envelope.

In one embodiment, the carrier frequency lies in the range of approximately 220 Hz to 400 Hz, facilitating stable capacitive coupling across the stratum corneum. The amplitude envelope may have a characteristic frequency in the range of approximately 0.1 Hz to 20 Hz. Such envelopes' behavior may be produced by amplitude variation, burst timing, duty-cycle variation, or related signal control methods.

The carrier signal provides efficient capacitive coupling into superficial tissue layers, while the envelope introduces temporal variation of the applied electric field conditions at time scales longer than the carrier period.

Biological tissues exhibit nonlinear electrical properties and capacitive behavior. As a result, higher-frequency components may be partially attenuated or transformed within tissue, while lower-frequency envelope components may contribute to temporal variation of local electrical conditions.

Accordingly, the combined use of a higher-frequency carrier and a lower-frequency amplitude envelope enables simultaneous control of coupling efficiency and temporal patterning of the applied electric field without requiring an increase in signal amplitude.

### Sub-Resonant Electric Field Operation

In certain embodiments, the piezoelectric membrane is driven at frequencies substantially below its mechanical resonance frequency (referred to herein as a deep sub-resonant regime), such that mechanical vibration of the membrane is minimal or negligible. For example, the electrical drive signal may have a frequency in a range of approximately 1 Hz to 60 Hz, which is significantly lower than a fundamental resonance frequency of the piezoelectric structure.

Under such conditions, the piezoelectric membrane does not function primarily as an acoustic transducer. Instead, electrical excitation produces time-varying electric fields that are capacitively coupled into adjacent tissue. Effective coupling of the electric field in this sub-resonant regime is facilitated by capacitive interactions between the piezoelectric membrane, one or more conductive members of the device, and the body of a user, including through a return path established by distributed capacitive coupling.

In this manner, the device is configured to produce displacement-current-dominated interaction with superficial tissue layers even in the absence of substantial mechanical displacement of the piezoelectric membrane.

In certain embodiments, the electrical drive signal comprises a non-sinusoidal waveform, including a rectangular or pulse waveform, whereby rapid transitions in the waveform inherently produce a plurality of harmonic frequency components, thereby enriching the spectral content of the electric field coupled into the tissue, and contributing to formation of a composite time-varying field envelope.

### Device Control and Signal Generation

In certain embodiments, operation of the device is controlled by a controller, such as a microcontroller (32), executing instructions stored in memory. The controller is configured to manage generation and timing of electrical signals applied to the piezoelectric membrane.

The controller may generate or control excitation signals having predetermined waveform shapes, frequencies, and temporal patterns. In some embodiments, signal generation functions that may otherwise be implemented using discrete hardware components are performed in software.

The controller may be configured to produce quasi-sinusoidal or sinusoidal excitation signals, including signals exhibiting frequency variation and amplitude variation as described herein. Signal conditioning operations, including shaping and filtering of generated waveforms, may be implemented to control spectral content of the excitation signal.

The controller may further manage operational parameters including selection of carrier frequency, frequency variation parameters, amplitude variation characteristics, and timing patterns such as duty cycle, burst operation, and repetition intervals.

User interaction with the device may be facilitated through an input-output interface, allowing selection of operating modes and adjustment of signal parameters. The controller may execute predefined or user-selectable programs corresponding to different operating conditions.

### Application and Field Interaction

The device may be positioned relative to the skin in different orientations to modify spatial characteristics of the projected electric field.

In a first orientation, the piezoelectric element and associated conductive members are arranged substantially parallel to the skin surface, producing a distributed electric field covering a relatively broad tissue area. In a second orientation, the element is arranged at an angle or substantially perpendicular to the skin surface, resulting in a more localized field distribution with increased spatial gradients.

The device may be operated in a non-contact mode at a distance from the skin, or in direct contact, including through an intervening dielectric layer such as clothing. The intervening material may define a localized dielectric interface that modifies capacitive coupling between the device and tissue.

Application of a time-varying drive signal to the piezoelectric membrane produces a spatially bipolar electric field at the tissue interface. Opposing polarities associated with different regions of the electrode structure generate lateral voltage differentials within superficial tissue, resulting in spatially varying electric field gradients.

Mechanical deformation of the piezoelectric membrane during operation produces lateral displacement of projected field maxima, resulting in dynamic spatial redistribution of the electric field across the skin surface. This effect constitutes spatial scanning of electric field coupling.

The combined effect of temporal variation and spatial displacement of the electric field produces controlled spatial inhomogeneity of field distribution within superficial tissue layers. Such spatiotemporal variation enables engagement of multiple localized tissue regions without substantial increase in penetration depth.

In embodiments employing floating electrode configurations, electric field interaction with tissue occurs primarily through capacitive coupling and displacement-current mechanisms.

### Spatial Field Shaping and Focusing Structures

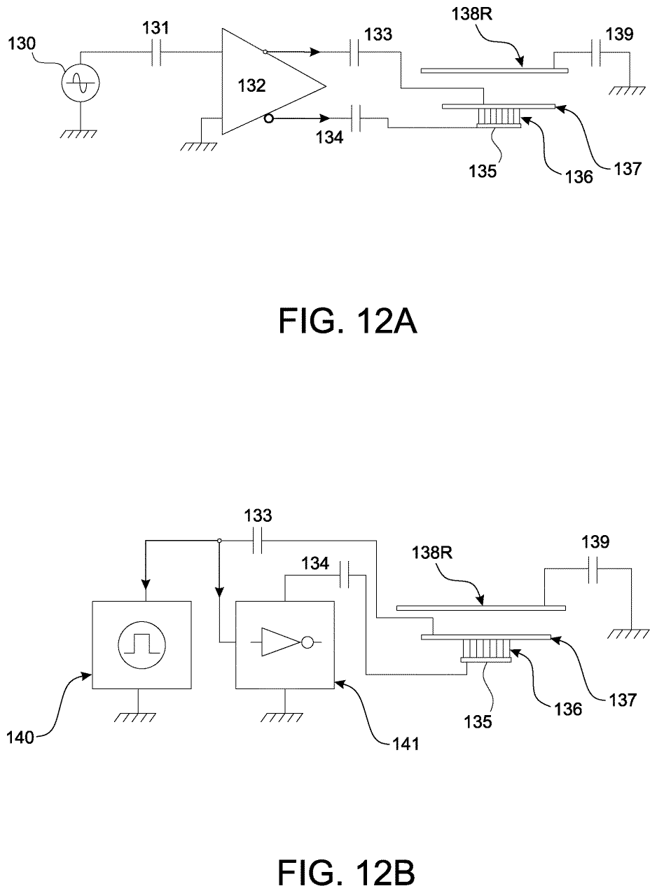

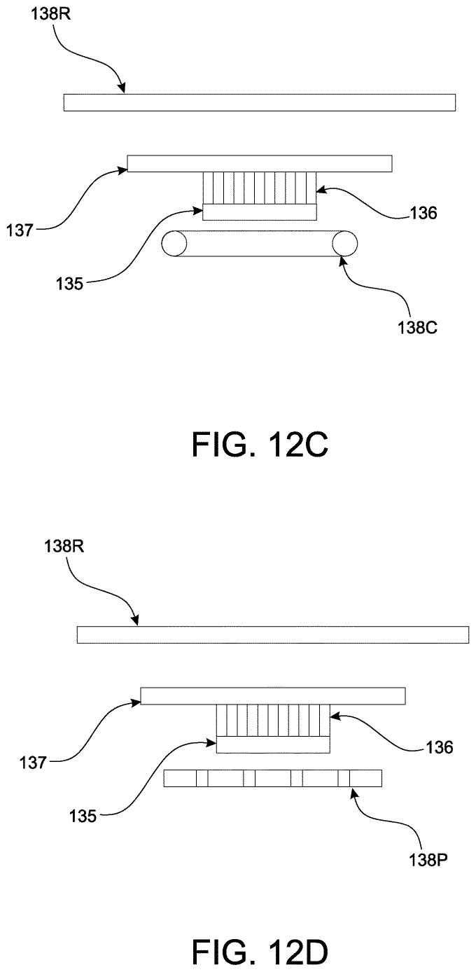

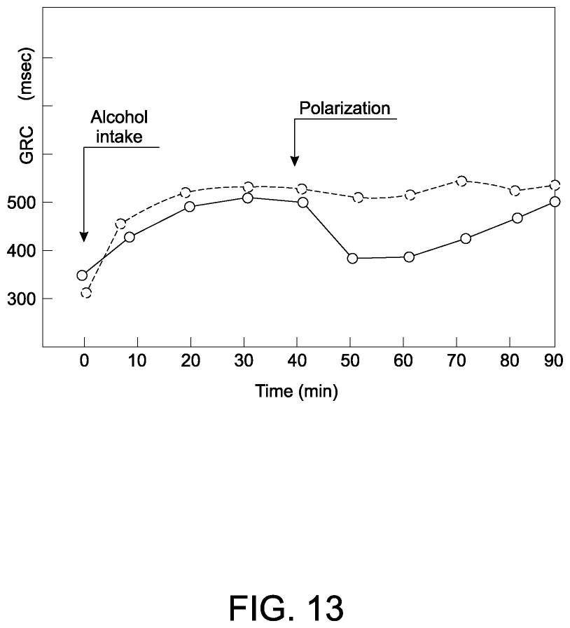

In addition to active spatial scanning mechanisms, the device may include one or more spatial field shaping structures configured to modify, concentrate, or redistribute the projection of the electric field onto the skin. Representative embodiments are illustrated in FIGS. 12C and 12D.

In certain embodiments, the piezoelectric membrane assembly (135-137) includes a rear conductive plate (138R) configured to influence field distribution and contribute to formation of a spatially bipolar electric field at the tissue interface.

In addition, one or more passive field-shaping elements may be positioned in front of the piezoelectric membrane.

In one example (FIG. 12C), a conductive ring (138C) is positioned coaxially with the piezoelectric layer (136) and disposed adjacent to the front surface of the membrane. The ring preferably has a diameter slightly greater than that of the piezoelectric layer such that it surrounds the peripheral region of the membrane, which is typically associated with increased electric-field divergence. The conductive ring modifies boundary conditions of the electric field and acts to redistribute field lines, thereby concentrating the projected field toward the central region of the membrane.

In another embodiment (FIG. 12D), the conductive ring is replaced by a conductive perforated plate (138P) positioned in front of and in close proximity to the piezoelectric membrane. The perforated plate includes a plurality of openings through which electric field lines pass, thereby redistributing the spatial pattern of the projected electric field and producing localized regions of enhanced field intensity.

The passive field-shaping structures may operate in a floating configuration. In certain embodiments, such structures are entirely electrically floating, having neither galvanic connection nor intentional capacitive coupling to the driving circuitry. In other embodiments, the structures are galvanically isolated but capacitively coupled to a reference node of the circuit, for example through one or more capacitors, thereby providing AC coupling while maintaining DC isolation. Such capacitive coupling may function as an ungrounded high-pass network.

The presence of such passive structures modifies the spatial distribution of electric field gradients and may increase local field intensity and gradient steepness, thereby enhancing spatial localization and density of displacement currents induced within superficial tissue layers.

### Analog Floating Bipolar Drive

FIG. 12A illustrates an analog implementation of a floating bipolar drive architecture.

An oscillator (130) is configured to generate a sinusoidal or quasi-sinusoidal signal, which is AC-coupled via capacitor (131) to a bipolar driving amplifier (132). The amplifier produces two output signals of substantially equal amplitude and opposite phase polarity.

Each amplifier output is coupled to the piezoelectric element assembly (135-137) through respective capacitors (133, 134). These capacitors provide AC coupling while blocking DC conduction, thereby electrically isolating the amplifier outputs from the device ground. As a result, the excitation applied across the piezoelectric element is galvanically isolated and electrically floating relative to the device power reference.

The electrode system further includes an auxiliary electrode (138) positioned adjacent to the piezoelectric element assembly. The auxiliary electrode is configured to perform two functions:
1. Spatial field shaping, modifying distribution and projection of the electric field generated by the piezoelectric element; and
2. Capacitive return-path participation, contributing to closure of a displacement-current loop through distributed capacitive coupling with the subject and surrounding environment.

The auxiliary electrode (138) is capacitively coupled to a circuit reference node via capacitor (139), thereby maintaining galvanic isolation while enabling AC interaction with the field distribution. In this manner, the auxiliary electrode participates in redistribution of electric field boundary conditions without establishing direct conductive contact.

In the illustrated configuration, all electrodes interacting with the subject are AC-coupled and galvanically isolated, forming a floating electrode system in which continuity of current is established through displacement currents and distributed capacitive coupling rather than through direct conductive injection.

### Digital Floating Bipolar Drive

FIG. 12B illustrates a floating bipolar drive configuration implemented using digital signal generation.

A digital oscillator (140) is configured to generate a periodic drive signal, for example a square-wave signal. The output of the oscillator is applied both directly and through an inverter (141), thereby producing two complementary signals having opposite phase states.

These complementary signals are capacitively coupled through capacitors (133, 134) to the piezoelectric element assembly (135-137). The coupling capacitors block DC conduction and maintain galvanic isolation from the device ground while permitting AC excitation of the piezoelectric structure. As a result, the drive applied across the piezoelectric element is electrically floating relative to the device power reference.

In certain embodiments, the digital drive signals may be further processed to approximate sinusoidal or quasi-sinusoidal waveforms, for example by filtering, pulse-width modulation, or controlled edge shaping, thereby enabling control over spectral content of the excitation signal.

An auxiliary electrode (138) is positioned adjacent to the piezoelectric element assembly and operates in a manner consistent with the embodiment of FIG. 12A. The auxiliary electrode is configured to:
1. Modify spatial distribution of the electric field, including focusing and redistribution of projected field lines; and
2. Participate in capacitive return-path formation, contributing to closure of the displacement-current loop through distributed coupling with the subject and surrounding environment.

The auxiliary electrode (138) is capacitively coupled to a circuit reference node via capacitor (139), thereby remaining galvanically isolated while participating in AC field redistribution.

Accordingly, the digital embodiment provides a functionally equivalent floating electrode system in which all electrodes interacting with the subject are AC-coupled and galvanically isolated, and in which current continuity is established through displacement currents and distributed capacitive coupling rather than through direct conductive injection.

### Functional Outcome of Floating Electrode Configuration

In the embodiments of FIGS. 12A and 12B, the electrode system comprising the piezoelectric element assembly (135-137) and auxiliary electrode (138) is maintained in an AC-coupled and galvanically isolated (floating) state relative to the device ground.

Such floating configuration enables spatially symmetric bipolar electric field projection at the tissue interface, reduces reference-dependent field distortion, and facilitates interaction with biological tissue through capacitive coupling and displacement-current mechanisms.

The floating electrode arrangement further enables control over electric field geometry and spatial distribution of induced displacement currents within superficial tissue layers

### Application and Use Configurations

The device is configured for application to a subject's body either in direct contact with the skin or in a non-contact configuration, including through an intervening dielectric layer such as clothing. The intervening material may define a localized dielectric interface that modifies capacitive coupling between the device and superficial tissue.

In certain embodiments, the dielectric layer has a thickness in a range of approximately 0.1 mm to 3 mm and may comprise textile materials including cotton, polyester, or blends thereof.

Field Interaction with Tissue

During operation, the device generates a time-varying electric field that is capacitively coupled into superficial tissue without direct galvanic contact.

The electric field is spatially non-uniform and varies over time, including dynamic spatial redistribution of field maxima across the skin surface. In certain embodiments, lateral displacement of field concentration regions may occur on a microscale, including on the order of approximately 10 μm to 500 μm.

Drive signals may include frequency components in a range of approximately 0.1 Hz to 500 Hz, including carrier components and lower-frequency modulation components. Applied voltages may be selected within a range sufficient to establish displacement-current interaction with superficial tissue while avoiding direct conductive current injection.

The spatiotemporally varying electric field may interact with superficial sensory structures by introducing spatial redistribution and temporal variation of local electric field gradients. Due to non-linear and threshold-dependent characteristics of biological signaling, such variations may influence sensory perception without requiring direct neural excitation.

### Example Wellness-Oriented Applications

In certain embodiments, the device may be used in sports, fitness, or general wellness contexts.

Applications may include using after physical exertion to support comfort and relaxation, or during rest periods to reduce perceived tension. In some embodiments, the device may be applied to localized skin regions associated with sensory discomfort, including irritation or itching.

In further embodiments, application may be directed to muscle groups to support relaxation of localized tension.

In certain implementations, the device may be used as part of general wellness routines intended to support perceived well-being.

All such applications are achieved through capacitive coupling of time-varying electric fields into superficial tissue without direct conductive current injection.

### Development of Capacitive Electric Field Neuromodulation

### Early Locus-Focused Investigations

Early investigations conducted by the present inventor evaluated the effect of externally applied electric field polarization directed to localized anatomical regions using non-conductive coupling.

These studies are reported in Kitov, Z., “Acupuncture-like Effect Produced by Electrical Field Focused on Specific Loci,” Proceedings of the Ninth Annual Conference of the IEEE Engineering in Medicine and Biology Society, 1987.

In these experiments, low-frequency electric fields were applied to defined loci using insulated electrode configurations that prevented direct galvanic current injection. Measurements of a General Responsiveness Coefficient (GRC), representing reaction time to external stimuli, were recorded under active and sham conditions.

Following induction of reduced responsiveness, application of localized electric field exposure was associated with a return of responsiveness toward baseline values, whereas sham conditions showed no comparable change.

These observations demonstrate that externally applied electric fields, coupled through non-conductive interfaces, can influence measurable functional parameters of a subject.

Without being bound to any specific biological mechanism, such effects are consistent with displacement-current-mediated polarization occurring under non-galvanic coupling conditions.

### Early Insulated Electrode Clinical Evaluation

Subsequent investigations conducted in collaboration with the Department of Neurosurgery at George Washington University evaluated insulated capacitive field stimulation systems, as described in Guthrie, B. et al., “Investigation of the Novel Electrostimulation Modality ‘Ortho-Plus 200’ as a Tool for the Control of Postoperative Pain,” GWU Medical Center Status Report, 1989-1990.

The evaluated systems employed electrodes electrically insulated from the skin, thereby promoting electric field coupling across a dielectric interface and avoiding direct conductive current injection.

Under these conditions, stimulation occurred below perceptible motor thresholds and did not produce observable muscle contraction.

Preliminary observational results indicated reduced narcotic usage in treated postoperative subjects relative to controls. These findings were exploratory and non-limiting.

The investigations further identified the importance of non-uniform electric field distribution, including spatial gradient components, in achieving functional effects.

Consistence with Bioimpedance Models

The foregoing observations are consistent with established models of tissue electrical properties.

The stratum corneum functions as a high-impedance dielectric layer, supporting capacitive coupling of externally applied electric fields under non-galvanic conditions, as described in Miranda et al.

Multilayer impedance models indicate that displacement currents dominate initial coupling into tissue, as described in Birgersson, (pages 32 to 52).

Computational analyses further demonstrate that tissue heterogeneity influences electric field localization and gradient formation.

Accordingly, the historical observations described above are consistent with superficial displacement-current interaction mechanisms under insulated electrode excitation.

### CONCLUSION

Accordingly, the foregoing description demonstrates that capacitive coupling modulation employing time-varying electric fields may be configured to produce controlled, non-galvanic interaction with superficial tissue.

By utilizing insulated interfaces, displacement-current-dominant excitation, and defined electric field geometries, the disclosed systems enable depth-constrained electrical interaction without requiring direct conductive current injection.

In certain embodiments, such interaction may influence superficial sensory and afferent pathways through spatial and temporal variation of electric field distribution.

The embodiments described herein are illustrative and not limiting. Variations in electrode geometry, field distribution, waveform characteristics, anatomical placement, and system configuration may be implemented without departing from the principles set forth herein.

The scope of the invention is defined by the appended claims and their equivalents.

### APPENDIX: EQUIVALENT CIRCUIT MODELING OF CAPACITIVE COUPLING

In the following expressions, j denotes the imaginary unit and ω=2πf, where f is frequency. The symbol ∥ denotes parallel impedance. Vertical bars |·| denote magnitude.

The following models are illustrative and non-limiting and are provided to assist in understanding capacitive coupling interaction with biological tissue.

### Equivalent Circuit Representation

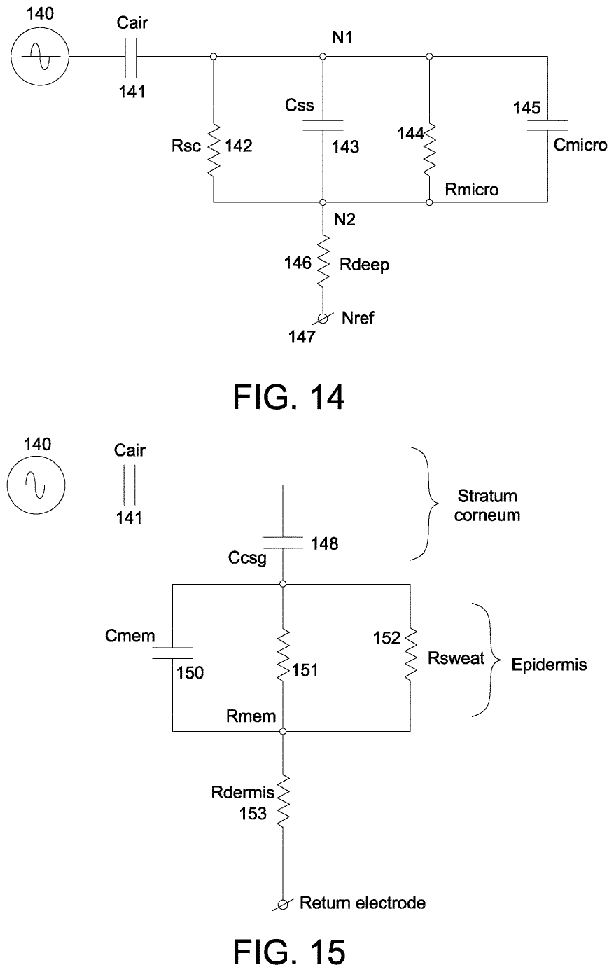

FIG. 14 illustrates a device configured to generate a time-varying electric field coupled across an intervening air gap to the skin surface (node N1).

The interface between the device and tissue is represented by a multilayer equivalent circuit comprising:
coupling capacitance C_air (141)
stratum corneum impedance Z_SC
deeper pathway resistance R_deep (146)
remote reference node N_ref (147)

The overall structure may be represented as:

C_air→Z_SC→R_deep→N_ref

This configuration reflects coupling through distributed capacitive and resistive pathways rather than direct conductive electrode contact.

### Stratum Corneum Model

The stratum corneum is represented as:
bulk resistance R_SC (142)
capacitance C_SC (143)
   with additional microchannel pathways:
R_micro (144)
C_micro (145)

The impedance is:

$${Z}_{SC} = ( {R}_{SC} ‖ \frac{1}{j ω {C}_{SC}} ) ‖ ( {R}_{micro} ‖ \frac{1}{j ω {C}_{micro}} )$$

### Viable Epidermis Model

FIG. 15 illustrates a subcircuit comprising:
membrane branch: R_mem (151), C_mem (150)
sweat pathway: R_sweat (152)
dermal resistance: R_dermis (153)

The impedance is:

$${Z}_{EPI} = ( {R}_{mem} ‖ \frac{1}{j ω {C}_{mem}} ) ‖ {R}_{sweat}$$

### Operating Interpretation

Application of a time-varying voltage across C_air produces displacement current into the superficial tissue network.

Current distribution is influenced by:
capacitive membrane pathways
resistive sweat duct pathways
deeper conductive return paths

### Representative Parameter Ranges (Non-Limiting)

R_SC: ~10 kΩ to several MΩ
C_SC: ~10 nF/cm² to 1 μF/cm²
R_micro: ~5 kΩ to 500 kΩ
C_micro: ~1 nF to 200 nF
C_mem: ~1 nF to 1 μF
R_mem: ~10 kΩ to 10 MΩ
R_sweat: ~5 kΩ to 500 kΩ
R_dermis: ~100Ω to 10 kΩ

### Example: Membrane Coupling Dominance

The ratio of membrane capacitive current to sweat pathway current is:

$$\vert \frac{{I}_{{C}_{mem}}}{{I}_{sweat}} \vert = 2 π {fC}_{mem} {R}_{sweat}$$

Membrane-dominant coupling occurs when:

$$f > \frac{1}{2 π {R}_{sweat} {C}_{mem}}$$

For example:
R_sweat≈50 kΩ
C_mem≈50 nF
threshold frequency ≈64 Hz

At higher frequencies, membrane-associated capacitive current may dominate over resistive pathways.

The above modeling framework is consistent with established bioimpedance descriptions of skin as a layered dielectric-conductive structure and supports interpretation of electric field coupling under non-contact or insulated conditions.

1. An electric field stimulation device, comprising:
a piezoelectric membrane including a piezoelectric ceramic layer bonded to a conductive metal plate,
an electrical drive circuit configured to apply a time-varying electrical signal of alternating polarity across the piezoelectric ceramic layer and the conductive metal plate,
wherein the piezoelectric membrane is arranged such that the ceramic layer faces a target tissue during use,
wherein the device is configured to capacitively couple an electric field into superficial tissue layers without galvanic electrical contact,
wherein the arrangement of the piezoelectric ceramic layer and the conductive metal plate produce a spatially bipolar electric field projection at a tissue interface, and
wherein the device further comprises at least one Spatial Field Shaping element positioned adjacent to the piezoelectric membrane and configured to selectively modify a spatial distribution of the bipolar electric field to concentrate field intensity at selected locations at the tissue interface.

2. The electric field stimulation device of claim 1, configured to operate selectively in:
(a) a floating drive mode in which all electrodes are galvanically isolated from a power supply and excited through capacitive coupling; and
(b) a referenced drive mode in which at least one electrode is conductively coupled to the power supply.

3. The electric field stimulation device of claim 1, wherein electric field interaction with the tissue is predominantly capacitive and characterized by displacement current flow across a non-conductive interface without direct galvanic current injection, thereby limiting conductive current injection and controlling penetration depth.

4. The electric field stimulation device of claim 1, wherein the electrical drive circuit comprising an output transformer having a secondary winding coupled to the piezoelectric membrane, such that the electrical drive signal applied to the piezoelectric membrane is electrically isolated from the power supply, and wherein the secondary winding of the output transformer includes a midpoint that functions as a floating reference potential.

5. The device of claim 4, further comprising a conductive member positioned within a device housing and electrically coupled to a floating reference potential of an output drive circuit, wherein the conductive member is positioned such that, during handheld operation, capacitive coupling between the conductive member and a body of a user, including capacitive coupling through a hand or arm of the user holding the device, provides a return path for displacement current.

6. The device of claim 5, wherein the conductive member is a conductive plate capacitively coupled to a floating reference potential via a first capacitor component, and wherein each terminal of the piezoelectric membrane is capacitively coupled to a respective terminal of a secondary winding of a transformer via corresponding capacitor components, such that all electrodes involved in electric field generation are galvanically isolated and capacitively coupled to the drive circuit.

7. The device of claim 5, wherein the body-coupled capacitive return path stabilizes the electric field distribution by reducing variability associated with uncontrolled capacitive coupling between the user's body and external ground or electrical power wiring, and wherein displacement current continuity through the return path enables stable and repeatable coupling of the electric field into superficial tissue layers while maintaining minimal conductive current flow and wherein at least a portion of the return path is established via a floating electrode.

8. An electric field stimulation device of claim 1, wherein the mechanical vibration of the piezoelectric membrane or associated conductive structure produces a time-varying spatial displacement of an electric field distribution, such that regions of maximum electric field intensity are dynamically shifted across the surface of the target tissue to sequentially stimulate spatially distinct microdomains.

9. The device of claim 8, wherein the mechanical vibration produces a lateral displacement of the projected electric field maxima across the tissue surface by a distance of at least 10 μm.

10. The device of claim 9, wherein the lateral displacement of the electric field maxima is sufficient to shift stimulation between adjacent microstructural targets within the tissue.

11. The device of claim 10, wherein the microstructural targets comprise sweat ducts, nerve endings, and cellular clusters having characteristic spacing in the range of 10 μm to 500 μm.

12. The device of claim 10, wherein the lateral displacement is at least one-half of the average spacing between adjacent microstructural targets.

13. The device of claim 5, wherein the conductive member is positioned vertically with respect to the piezoelectric membrane.

14. The device of claim 5, wherein the conductive member is positioned in parallel with respect to the piezoelectric membrane.

15. The device of claim 2, wherein the floating drive mode is arranged such that continuity of displacement current is established through distributed capacitive coupling between floating electrodes of the device and the body of a user during use, including capacitive coupling through a hand or arm of the user holding the device, wherein at least a portion of the displacement current return path is provided via a floating electrode, thereby stabilizing the electric field distribution without direct conductive current injection into tissue.

16. The device of claim 1, wherein the Spatial Field Shaping element comprises a conductive ring positioned adjacent to and at least partially surrounding the electrodes, wherein the conductive ring is arranged coaxially with the conductive electrodes, and wherein the conductive ring redistributes fringe electric field lines to concentrate the electric field toward a central region of the target tissue.

17. The device of claim 16, wherein the conductive ring is dimensioned and positioned to overlap an edge region of the conductive electrode corresponding to a region of enhanced fringe electric field and increased field gradient.

18. The device of claim 1, wherein the Spatial Field Shaping element comprises a conductive sheet including a plurality of apertures, wherein the conductive sheet is positioned between the conductive electrodes and the target tissue, wherein the apertures are arranged such that electric field lines are concentrated through the apertures, forming localized regions of increased field intensity.

19. The device of claim 1, wherein the Spatial Field Shaping element comprises both the conductive ring and the perforated conductive sheet, wherein the conductive ring and perforated sheet cooperatively shape the electric field distribution to produce enhanced spatial focusing on the target tissue interface.

20. The device of claim 1, wherein the Passive Spatial Field Shaping element comprises a conductive cylindrical structure positioned adjacent to and at least partially surrounding a peripheral region of the conductive electrode, the cylindrical structure having a longitudinal axis oriented substantially perpendicular to a plane of the conductive electrode.

21. The device of claim 1, wherein the electrical drive circuit is configured to produce a time-varying electric field having a spectral distribution comprising a plurality of frequency components distributed about a central frequency,
wherein at least a portion of said frequency components are separated from the central frequency by integer multiples of a modulation frequency,
and wherein interaction of said frequency components produces a composite electric field envelope at the tissue interface,
wherein the electrical drive circuit is configured to produce both (i) variation of instantaneous frequency and (ii) variation of amplitude of the time-varying electric field such that said composite electric field envelope is formed.

22. The device of claim 21, wherein the plurality of frequency components includes at least 50 distinct frequency components, and wherein amplitudes of at least a portion of said frequency components are sufficient to produce a temporally varying displacement current within superficial tissue layers corresponding to said composite electric field envelope,
wherein said envelope varies at a frequency lower than said central frequency.

23. The device of claim 1, wherein the electrical drive circuit is configured to apply a time-varying electrical signal having a frequency substantially below a mechanical resonance frequency of the piezoelectric membrane such that mechanical vibration of the piezoelectric membrane is negligible,
and wherein the device is configured to capacitively couple an electric field into the tissue under said conditions through interaction of the piezoelectric membrane with at least one conductive member providing a return path for displacement current.
